(19) United States
(12) Patent Application Publication
(10) Pub. No.: US 2020/0187489 A1
(43) Pub. Date: Jun. 18, 2020

(54) MATRICES AND METHODS FOR STORAGE AND STABILIZATION OF BIOLOGICAL SAMPLES COMPRISING VIRAL RNA

(71) Applicant: GenTegra, LLC, Pleasanton, CA (US)

(72) Inventors: Shanavaz Nasarabadi, Pleasanton, CA (US); Raymond Lenhoff, Pleasanton, CA (US)

(21) Appl. No.: 16/220,778

(22) Filed: Dec. 14, 2018

(51) Int. Cl. A01N 1/02; G01N 1/30; C12Q 1/6806

(52) U.S. Cl. CPC A01N 1/0231; G01N 2001/307; C12Q 1/6806; G01N 1/30

(57) ABSTRACT

Matrices and methods of stabilizing and storing biological samples containing nucleic acids are provided. The present application in particular provides systems and methods for stabilizing RNA and especially viral RNA in raw samples, where the stabilization and storage occur before additional processing, isolation, and analytical steps have taken place.

### GOVERNMENT SPONSORSHIP

This invention was made with government support under Grant Contract Number 12244564 awarded by the NIAID division of the NIH. The government has certain rights in the invention.

### FIELD OF THE INVENTION

The invention relates generally to matrices and methods for stabilizing and storing biological samples containing nucleic acids, particularly RNA and especially viral RNA, wherein the stabilization and storage occurs before additional processing, isolation, and analytical steps have taken place.

### BACKGROUND OF THE INVENTION

Many industries require effective methods and systems of stabilizing and storing fully intact nucleic acid sequences obtained from raw samples, such as genomic DNA and RNA obtained from whole blood and plasma. For the pharmaceutical, medical, law enforcement, military, and other molecular research industries, it is highly desirable to store and have access to many biological samples containing nucleic acids. Stabilization and storage methods must maintain long-term sample integrity to prevent the loss of materials which are often irreplaceable or otherwise difficult to acquire. Further, to allow facilities to obtain and store a high volume of nucleic acids, such stabilization and storage means must be easily transportable and allow for a streamlined processing and handling of a high volume of samples, while not requiring complicated and expensive maintenance.

Existing methods of stabilizing and storing nucleic acids obtained from raw samples suffer from high cost and/or poor sample integrity. For example, the standard method for storage and preservation of RNA is at ultra-low temperatures, usually through the use of liquid nitrogen and/or freezers. However, shipping samples in this manner is expensive, hazardous, and often results in the samples being subject to high variations in temperature during the shipping process. Alternatively, some existing methods turn to desiccation. Although desiccated samples are less expensive to ship, desiccated samples require extensive laboratory preparation in order to stabilize the samples. This preparation is usually not feasible when the nucleic acids to be stabilized are in a raw sample, i.e. found in whole blood or plasma, as the sample must be stabilized and stored before nucleic acid isolation and additional processing.

The problem is further exacerbated by the fact that nucleic acids, particularly RNA, can degrade very quickly if stored in improper conditions. RNA is especially labile; it can spontaneously degrade even in an aqueous medium. As a result, the storage of viral RNA poses a significant challenge beyond that of most nucleic acids. This has been discussed in the literature. For example, Garcia-Lerma et al of CDC describes the difficulties of storing viruses in dried plasma spots and dried blood spots at ambient. Garcia-Lerma et al. Rapid decline in the efficiency of HIV drug resistance genotyping from dried blood spots (DBS) and dried plasma spots (DPS) stored at 37 C and high humidity, Journal of Antimicrobial Chemotherapy 64(1):33-6 (May 2009). Consequently, there is a need in the field to develop additional nucleic acid storage materials and systems.

### SUMMARY OF THE INVENTION

Therefore, it is a principal object, feature, and/or advantage of the present invention to provide systems and methods for the long-term preservation of nucleic acids from raw samples, such as DNA and RNA, wherein the system preserves sample integrity at low cost under a variety of temperatures, humidity levels, and conditions.

Many industries require inexpensive, user-friendly, long-term storage systems for nucleic acids. Most biological and molecular research applications need to be able to store and analyze a high volume of samples, especially for high throughput screening/analysis. If the samples require complicated cooling or stabilization means, the systems are too costly. However, if the storage system requires too much labor or preparation, it is too time consuming and/or laborious to analyze a very high volume of samples. Similarly, in the contexts of epidemiology and laboratory disease testing, the loss of sample integrity during specimen transport or storage can lead to false-negative diagnostic results. Finally, in law enforcement and military applications, the loss of sample integrity can result in the loss of nucleic acid material that is irreplaceable, such as trace samples collected in the course of criminal investigation. Furthermore, in many applications and particularly in law enforcement, a high recovery of the sample material is critical, as even a 90% recovery may yield too little material to analyze.

The need for effective stabilization and storage systems is especially challenging with respect to nucleic acids. RNA in particular is especially labile, and can degrade very quickly. Aqueous RNA can be degraded by spontaneous phosphodiester bond cleavage as a result of acid or base catalyzed transesterification from the intramolecular nucleophilic attack of the 2′ hydroxyl group on the phosphorous atom. Additionally, ribonuclease (RNases) which enzymatically degrade aqueous RNA are virtually ubiquitous in all cells, and pose a constant threat of contamination and degradation of purified RNA.

These problems are particularly prescient for blood. Blood samples are often collected at one site and processed for isolation elsewhere. Under these circumstances, for example, the RNA must be stabilized prior to shipping and RNA purification. The first hurdle is that blood has a complex cellular composition. There are several sources of RNA in blood. Leukocytes contain RNA, but comprise <1% of the cell mass of blood. Additionally, circulating RNA can be found in plasma. In some contexts, the blood may contain viral RNA desirable for extraction, such as HIV-1. However, these quantities of RNA are extremely low relative to the overall cell mass of whole blood. More than 99% of the cellular blood fraction is composed of red blood cells, including immature reticulocytes, which contain high levels of globin mRNA. Globin mRNA can comprise the detection of other specific mRNAs from leukocytes, and can degrade leukocyte RNA, circulating RNA, and viral RNA.

Existing storage systems combat this problem by storing RNA at between −20° C. to −80° C., or in liquid nitrogen to provide protection from degradative reactions. Significantly, existing methods cannot effectively stabilize and store RNA—especially RNA from whole blood or plasma—at room temperature. Further, existing low-temperature methods are extremely costly, as shipping RNA on dry is expensive, requires special handling, is subject to air travel regulations, is time sensitive, and requires a high cost of storage upon arrival to a destination in terms of the cost to run and maintain ultra-low temperature (ULT) freezers. Further, even when it is economically feasible to use cold temperature or liquid nitrogen storage, these methods are not failsafe. Power outages, natural disasters, shipping accidents, and machine malfunction, to name a few, have resulted in the loss of millions of dollars of biomolecular samples. Other available storage systems including dry storage and aqueous storage media generally require additional processing steps, both to prepare the sample for storage and to recover the sample from its storage state. These methods are often costly and/or time-consuming, reducing the feasibility of processing and handling a high volume of samples efficiently.

It is therefore an object of the present application to provide systems and methods for the inexpensive, long-term, and effective storage of nucleic acids, particularly RNA, at room temperature.

It is an object of the present application to provide systems and methods for the inexpensive, long-term and effective storage of viral and circulating RNA from whole blood and plasma.

It is a further object of the present application to provide such systems and methods, wherein the systems and methods are capable of storing nucleic acids, particularly viral and circulating RNA, at room temperature.

It is a further object of the present application to provide systems and methods for the preservation of nucleic acids in a raw sample using a matrix (e.g. a solid-state matrix) comprising one or more metal chelators, a hydroxyl radical scavenger, and a cell separation reagent.

In other embodiments, the composition is a matrix (e.g. a solid-state matrix) comprising one or more metal chelators, a hydroxyl radical scavenger, a singlet oxygen quencher, an RNase inhibitor and a stabilizer. In some embodiments, the stabilizer is a cell separation reagent. In an embodiment, two metal chelators are used, and in further embodiments the two metal chelators comprise citric acid and an aminocarboxylate. In some embodiments, the hydroxyl radical scavenger comprises mannitol, the singlet oxygen quencher comprises cysteine, the RNase inhibitor comprises ATA, and the cell separation reagent comprises a polyethylene glycol.

According to an aspect of the present application, the composition stabilizes and stores a sample. In an embodiment, the sample is viral RNA provided as part of a sample of whole blood or plasma, and the viral RNA is stored for at least five days. In an aspect, the viral RNA is stored on a paper carrier as dried blood spots (DBS) or dried plasma spots (DPS). In a further aspect, the composition stabilizes and stores viral RNA at a temperature between about 20° C. and about 60° C., including ambient temperature.

In another aspect, the present invention provides for a kit for stabilizing and storing viral RNA, wherein the kit includes a composition comprising one or more metal chelators, a hydroxyl radical scavenger, a singled oxygen quencher, an RNase inhibitor, and a cell separation reagent; one or more carriers; and one or more carriers. In an embodiment, the composition is combined with a sample comprising viral RNA, the sample is then held in the one or more carriers, and the sample is sealed by the one or more closures. According to an aspect of the present application, the composition of the kit stabilizes viral RNA for at least five days, and stabilizes viral RNA at an ambient temperature.

In an embodiment, the one or more carriers of the kit may comprise one or more vials, one or more wells, paper, and/or a cotton swab. The kit may further comprise an additional container for housing the composition and sample held in the one or more carriers and sealed by the one or more closures. In an aspect, the additional container comprises a box and/or an envelope. According to an embodiment, the kit may further comprise a pre-addressed mailing label.

In an aspect, methods of using the composition and/or kit are provided. In particular, the present application provides a method of using a kit for stabilizing and storing viral RNA, the method comprising providing a composition comprising one or more metal chelators, a hydroxyl radical scavenger, a singled oxygen quencher, an RNase inhibitor, and a cell separation reagent, wherein the composition stabilizes viral RNA for at least five days, and wherein the composition stabilizes viral RNA at an ambient temperature; collecting one or more raw samples; mixing the one or more raw samples with the composition in one or more carriers; and sealing the mixture in the carrier with closures.

In an aspect, the method further comprises the step of placing the sealed mixture in an additional container for housing the one or more carriers. The method may also comprise the step of adding protective materials to the additional container, wherein the protective materials comprise protective foam, packing peanuts, and/or shredded paper filler. Further, the method may also comprise the step of applying a pre-addressed mailing label to the additional container, and shipping the kit.

In a further aspect, the present application provides for a method of making and using the composition for stabilizing and storing viral RNA. The method may comprise combining one or more metal chelators, a hydroxyl radical scavenger, a singlet oxygen quencher, an RNase inhibitor, and a cell separation reagent. The composition may be provided as a concentrate or diluted using a suitable solvent. In a further aspect, a method of using the composition is provided, the method comprising combining the composition with a sample to form a mixture and/or matrix (depending on the phase of the composition and sample), and placing the mixture and/or matrix into one or more carriers.

The composition may be provided as a liquid or a solid, and may be dehydrated or rehydrated as needed during the method of use. For example, the composition may be first provided as a liquid and subsequently dehydrated for the storage of a sample. The composition may be embedded in, saturated on, or may otherwise inundate a solid medium, such as paper or any other suitable matrix. In an aspect, the matrix and/or paper encapsulates, captures, and/or suspends the sample, facilitating stable storage of the sample.

In a further aspect, stabilization and storage of the sample as described herein occur before additional processing, isolation, and/or analytical steps have taken place, thus enabling the stabilization and storage of a raw sample.

Additional aspects and details of the invention will be evident from the detailed description that follows.

### BRIEF DESCRIPTION OF THE DRAWINGS

FIG. 1 depicts the stabilization of HIV-1 virus in whole blood and in plasma on paper in the form of dried blood spots (DBS) and dried plasma spots (DPS) according to the compositions of the present application.

FIG. 2 depicts the stabilization of HIV-1 virus in whole blood and in plasma in solution according to the compositions of the present application.

FIG. 3 shows the stabilization of HIV-1 virus in whole blood on paper in the form of dried blood spots over the course of five days; FIG. 3 compares paper treated according to the compositions of the present application to control, i.e. traditional storage methods.

### DETAILED DESCRIPTION OF THE PREFERRED EMBODIMENTS

The embodiments of this invention are not limited to particular systems and methods for stabilizing and storing raw samples containing nucleic acids, particularly whole blood and plasma samples, which can vary. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (e.g. 1 to 5 includes 1, 1.5, 2, 2¾, 3, 3.80, 4, and 5).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts.

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

The terms “nucleic acid,” “oligonucleotide” and “polynucleotide” may be used interchangeably and encompass DNA, RNA, cDNA, whether single stranded or double stranded, as well as chemical modifications thereof and artificial nucleic acids (e.g., PNA, LNA, etc.). The source of the nucleic acids may vary, including but not limited to RNA derived from whole blood and plasma, especially viral RNA.

The terms “matrix,” “dry state,” and “solid-state matrix” as used herein refer to cellulose paper that has been impregnated with the stabilizing solution according to the present application.

The terms “stabilize” and “preserve” as used herein mean to render resistant to hydrolytic damage, oxidative damage, irreversible denaturation (unfolding or loss of secondary or tertiary structure), mechanical damage due to shearing or other force, and other damage. This resistance to damage also results in a retention of function and maintenance of integrity of a sample. Retention of function which is preserved and stabilized may include, without limitation, a pair of forward and reverse primers retaining their ability to prime amplification of a target polydeoxyribonucleotide or a target nucleic acid (e.g., genetic) locus; a reverse transcription primer retaining its ability to prime reverse transcription of a target polyribonucleotide; a biological sample retaining its biological activity or its function as an analyte in an assay, or components in the biological sample retaining their biological activity or their function as analytes in an assay; and bacterial cells retaining their infectivity in an appropriate medium (e.g., an agar medium or a fluid culture), or viral particles retaining their infectivity in an appropriate medium (e.g., a natural fluid or a laboratory cell culture).

As used herein, the terms “raw sample,” “raw material,” “whole sample” and “whole material” refer to a basic substance in its natural, modified, or semi-processed state wherein the material is not yet fully processed or prepared. The raw samples of the present application generally contain wholly or a high quantity of intact cells, i.e. cells that have not yet been intentionally lysed. Although some cells in a raw sample may be ruptured due to natural causes or the state of the sample upon collection, a raw sample according to the present application does not contain cells intentionally ruptured, or otherwise processed or prepared.

As used herein, the term “lysis” refers to the breaking down of the cell, often by viral, enzymatic, or osmotic reactions that comprises cell wall integrity. Cell lysis is used to break open cells to avoid shear forces that would otherwise denature or degrade sensitive proteins, DNA, RNA, and other components.

As used herein, the term “whole blood” means blood having none of the constituent components removed or intentionally separated. Whole blood contains, for example, red cells, white cells, and platelets suspended in blood plasma. Whole blood generally comprises approximately 55% plasma, 45% red blood cells, and <1% white blood cells and platelets. The whole blood may include components endemic to whole blood, and the whole blood may also include components nonnative to whole blood, including but limited viral, bacterial, pharmaceutical or other microorganism material such as HIV, hepatitis B, hepatitis C, etc.

As used herein, the term “plasma” references the liquid portion of blood which, when part of whole blood, suspends red and white blood cells and platelets. Blood plasmas generally contains about 92% water, 7% vital proteins (e.g. albumin, gamma globulin, and anti-hemophilic factor), and 1% mineral salts, sugars, fats, hormones and vitamins. The term “plasma” as used herein can refer to plasma occurring as part of whole blood, and/or it can refer to plasma separated from whole blood. The term “plasma” also encompasses all plasma derivatives, whether the derivatives occur within the plasma or have been separated from the plasma via fractionation. The plasma derivatives may be components endemic to plasma, including but not limited to Factor VIII Concentrate, Factor IX Concentrate, Anti-Inhibitor Coagulation Complex (AICC), Albumin, Immune Globulins, Anti-Thrombin III Concentrate, Alpha 1-Proteinase Inhibitor Concentrate. The plasma derivatives may also be components nonnative to plasma, including but limited viral, bacterial, pharmaceutical or other microorganism material such as HIV, hepatitis B, hepatitis C, etc. Plasma may further include circulating RNA and other circulating genetic or other biomarker materials.

As used herein, the terms ambient temperature” or “room temperature” refers to a temperature range from about 18° C. to about 27° C., or from about 20° C. to about 25° C., or from about 22° C. to about 40° C. In other embodiments, the term “ambient temperature” or “room temperature” refers to a temperature of about 18° C., 19° C., 20° C., 21° C., 22° C., 23° C., 24° C., 25° C., 26° C. or 27° C. In certain embodiments, the term “ambient temperature” or “room temperature” refers to a temperature of about 22° C., 37° C., 39° C. or 42° C.

### Compositions

The compositions of the present application may be used to stabilize and store one or more raw samples, particularly samples comprising viral RNA. The compositions of the present application are capable of inhibiting and/or mitigating undesirable contact between the raw sample (and components therein) and various contaminants or potential sources of degradation.

In some embodiments, the compositions of the present application are inert with respect to the raw samples (and components therein). As used herein, “inert” means that the inorganic compound either does not bind to one or more types of samples or binds reversibly such that the raw samples are not degraded as a result of such binding. Further, in an embodiment, the compositions of the present application are inert with respect to one or more downstream methods that may be used to analyze the raw samples and components therein. In this context, “inert” means that the presence of the compositions of the present application together with a raw sample does not reduce the rate of the downstream methods of analysis by more than 50% and does not significantly reduce the fidelity of the method. Exemplary methods of analysis may include, without limitation, nucleic acid transcription and/or amplification (e.g., reverse transcription, PCR, real time PCR, etc.), endonuclease digestion (e.g., reactions involving type II endonucleases, such as EcoRI, BamHI, HindIII, NotI, SmaI, BglII, etc.), cloning techniques (e.g., ligation), protein digestion (e.g., reactions involving proteinases such as proteinase K, trypsin, chymotrypsin, savinase, etc.), microarray analysis (e.g., of nucleic acids or proteins), immunoassays (e.g., immunoprecipitation, ELISA, etc.), mass spectroscopy, or any combination thereof. In certain embodiments, the inorganic compound is inert upon dilution (e.g., dilution by a factor of 2, 3, 4, 5, 6, 7, 8, 9, 10, 15, 20, or more).

In an embodiment, the components in the composition of the present application may also be water soluble. As used herein in this context, “water soluble” means that the inorganic compound has a solubility in water, at 25° C., of 1.0 mg/ml or greater. In certain embodiments, the inorganic compound has a solubility in water, at 25° C., of at least 1.5 mg/ml, 2.0 mg/ml, 3.0 mg/ml, 4.0 mg/ml, 5.0 mg/ml, 7.5 mg/ml, 10 mg/ml, 15 mg/ml, 20 mg/ml, 25 mg/ml, 30 mg/ml, 35 mg/ml, 40 mg/ml, 50 mg/ml, 60 mg/ml, 70 mg/ml, 80 mg/ml, 90 mg/ml, 100 mg/ml, 125 mg/ml, 150 mg/ml, 200 mg/ml, or greater. In certain embodiments, the inorganic compound can be easily solubilized in water. For example, in certain embodiments, the inorganic compound can be solubilized in water, at 25° C., in 75, 60, 50, 40, 30, 25, 20, 15, 10, 9, 8, 7, 6, 5, 4, 3, 2, or fewer minutes. In other embodiments, the inorganic compound can be solubilized in water, at 25° C., in 7, 6, 5, 4, 3, 2, 1.5, or fewer hours. In certain embodiments, the inorganic compound can be solubilized in water, at 25° C., with or without the use of agitation (e.g., pipetting, shaking, or vortexing).

The compositions of the present application may comprise: one or more metal chelators, a hydroxyl radical scavenger, a singlet oxygen quencher, an RNase and/or

DNase inhibitor, a cell separation reagent, and additional ingredients.

### Metal Chelator

In some embodiments, the composition contains one or more metal chelators. In an embodiment, the composition contains two or more metal chelators. As used herein, a “metal chelator” is a compound that forms two or more bonds with a single metal ion. In certain embodiments, the one or more metal chelators chelate at least one type of metal ion selected from the group consisting of magnesium ions, chromium ions, manganese ions, iron ions, cobalt ions, nickel ions, copper ions, zinc ions, lead ions, or any combination thereof. In certain embodiments, the one or more metal chelators chelate at least one type of metal ion and inhibit metal-dependent reactions between such ions and raw sample present in the composition. In certain embodiments, the one or more metal chelators chelate at least one type of metal ion and prevent such ions from degrading the raw sample (i.e. cells, components within the cells such as nucleic acids, and other materials of the raw sample) present in the composition. In preferred embodiments, the one or more metal chelators chelate magnesium ions and/or manganese ions and inhibit metal dependent reactions between such ions and biomolecules present in the composition. In other preferred embodiments, the one or more metal chelators chelate magnesium ions and/or manganese ions and prevent such ions from degrading biomolecules present in the composition.

Examples of suitable metal chelators include without limitation boric acid, aurintricarboxylic acid (ATA) and salts thereof [e.g., triammonium aurintricarboxylate (aluminon)], borate, citric acid, citrate, salicylic acid, salicylate, 1,2-bis(o-aminophenoxy)ethane-N,N,N′,N′-tetraacetic acid (BAPTA), diethylene triamine pentaacetic acid (DTPA), ethylenediaminetetraacetic acid (EDTA), ethylene glycol tetraacetic acid (EGTA), glycoletherdiaminetetraacetic acid (GEDTA), N-(2-hydroxyethyl)ethylenediamine-N,N′,N′-triacetic acid (HEDTA), nitrilotriacetic acid (NTA), 2,2′-bipyridine, o-phenanthroline, triethanolamine, and analogs, derivatives and salts thereof.

In an embodiment, the composition is substantially free of boric acid.

The one or more metal chelators may be present in the composition from about 1.5 mM to about 300 mM, preferably between about 150 mM to about 250 mM, preferably between about 160 mM to about 220 mM, and more preferably between about 175 mM to about 200 mM.

### Hydroxyl Radical Scavenger/Oxygen Radical Scavenger

The composition may comprise a hydroxyl radical scavenger/oxygen radical scavenger. These scavengers are capable of inhibiting undesirable contact between the raw sample (and components therein) and various contaminants or potential sources of degradation. Hydroxy radical scavengers can in particular protect against the effects of oxygen.

Examples of suitable hydroxyl radical scavengers include, but are not limited to mannitol and other sugar alcohols such as erythritol, sorbitol and xylitol, azides, cysteine, dimethylsulfoxide, histidine, salicylic acid, salicylate, monosaccharides, disaccharides (e.g., cellobiose, lactose, maltose, sucrose, and trehalose), complex sugars, and analogs, derivatives and salts thereof.

Examples of suitable oxygen radical scavengers include, but are not limited to, sugar alcohols (e.g., erythritol, mannitol, sorbitol, and xylitol), monosaccharides (e.g., hexoses, allose, altrose, fructose, fucose, fuculose, galactose, glucose, gulose, idose, mannose, rhamnose, sorbose, tagatose, talose, pentoses, arabinose, lyxose, ribose, deoxyribose, ribulose, xylose, xylulose, tetroses, erythrose, erythrulose, and threose), disaccharides (e.g., cellobiose, lactose, maltose, sucrose, and trehalose), complex sugars (e.g., trisaccharides, kestose, isomaltotriose, maltotriose, maltotriulose, melezitose, nigerotriose, raffinose, tetrasaccharides, stachyose, fructo-polysaccharides, galacto-polysaccharides, mannan-polysaccharides, gluco-polysaccharides, glycogen, starch, amylose, amylopectin, dextrin, cellulose, glucans, beta-glucans, dextran, fructans, inulin, glucosamine polysaccharides, chitin, aminoglycosides, apramycin, gentamycin, kanamycin, netilmicin, neomycin, paromomycin, streptomycin, tobramycin, glycosaminoglycans (mucopolysaccharides), chondroitin sulfate, dermatan sulfate, keratan sulfate, heparin, heparan sulfate, and hyaluronan), and analogs, derivatives and salts thereof.

The oxygen radical scavenger/hydroxyl radical scavenger may be present in the composition from about 100 mN to about 300 mM, preferably between about 150 mM to about 250 mM, and more preferably between about 175 mM to about 225 mM.

### Singlet Oxygen Quencher

A singlet oxygen quencher is capable of inhibiting undesirable contact between the raw sample (and components therein) and various contaminants or potential sources of degradation. Singlet oxygen quenchers can in particular protect against the effects of oxygen.

Examples of suitable singlet oxygen quenchers include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, L-selenocysteine, S-[2-(4-pyridyl)ethy]-L-cysteine, S-diphenylmethyl-L-cysteine, S-trityl-homocysteine, L-cysteine, S-ally-L-cysteine sulfoxide, S-aminoethyl-L-cysteine), phenolic compounds (e.g., tyrosine and derivatives thereof), aromatic acids (e.g., ascorbate, salicylic acid, and derivatives thereof), azides such as sodium azide, tocopherol and related vitamin E derivatives, and carotene and related vitamin A derivatives.

The singlet oxygen quencher may be present in the composition from about 100 mM to about 250 mM, preferably between about 150 mM to about 225 mM, and more preferably between about 175 mM to about 200 mM.

### RNase Inhibitors and DNase Inhibitors

Depending on the components of interest within the raw sample, the composition may comprise one or more RNase and/or DNase inhibitors. Suitable inhibitors may include, without limitation, aurintricarboxylic acid (ATA) and salts thereof [e.g., triammonium aurintricarboxylate (aluminon)], boric acid, borate, citric acid, citrate, salicylic acid, salicylate, 1,2-bis(o-aminophenoxy)ethane-N,N,N′,N′-tetraacetic acid (BAPTA), diethylene triamine pentaacetic acid (DTPA), ethylenediaminetetraacetic acid (EDTA), ethylene glycol tetraacetic acid (EGTA), glycoletherdiaminetetraacetic acid (GEDTA), N-(2-hydroxyethyl)ethylenediamine-N,N′,N′-triacetic acid (HEDTA), nitrilotriacetic acid (NTA), 2,2′-bipyridine, o-phenanthroline, triethanolamine, mammalian ribonuclease inhibitor proteins [e.g., porcine ribonuclease inhibitor and human ribonuclease inhibitor (e.g., human placenta ribonuclease inhibitor and recombinant human ribonuclease inhibitor adenosine 5′-pyrophosphate, 2′-cytidine monophosphate free acid (2′-CMP), 5′-diphosphoadenosine 3′-phosphate (ppA-3′-p), 5′-diphosphoadenosine 2′-phosphate (ppA-2′-p), leucine, oligovinysulfonic acid, poly(aspartic acid), tyrosine-glutamic acid polymer, 5′-phospho-2′-deoxyuridine 3′-pyrophosphate P′→5′-ester with adenosine 3′-phosphate (pdUppAp), and analogs, derivatives and salts thereof.

The RNase and/or DNase inhibitors may be present in the composition from about 0.1 mM to about 10 mM, preferably between about 0.5 mM to about 7 mM, and more preferably between about 1 mM to about 5 mM.

### Stabilizers

In some embodiments, the composition comprises one or more stabilizers. In an embodiment, the composition comprises two or more stabilizers. As used herein, a “stabilizer” is any agent capable of protecting nucleic acids, particularly nucleic acids occurring in a raw sample, from damage during storage. This may include without limitation, for example circulating RNA, viral RNA, DNA, and others.

In a preferred embodiment the stabilizer comprises a cell separation reagent. In a preferred embodiment, the cell separation reagent is polyethylene glycol. Suitable examples of cell separation reagents include, without limitation, polyethylene glycol 200 (PEG 200), polyethylene glycol 300 (PEG 300), polyethylene glycol 400 (PEG 400), polyethylene glycol 540 (PEG 540), polyethylene glycol 600 (PEG 600), polyethylene glycol 1000 (PEG 1000), polyethylene glycol 1450 (PEG 1450), polyethylene glycol 3350 (PEG 3350), polyethylene glycol 4000 (PEG 4000), polyethylene glycol 4600 (PEG 4600), polyethylene glycol 8000 (PEG 8000), Carbowax MPEG 350, Carbowax MPEG 550, Carbowax MPEG 750, and others.

The stabilizer may be present in the composition from about 35 wt. % to about 65 wt. %, preferably between about 40 wt. % to about 60 wt. %, and more preferably between about 45 wt. % to about 55 wt. %.

### Additional Ingredients

In some embodiments, the compositions can optionally contain one or more additional ingredients. For example, an antimicrobial agent, an organic or inorganic dye, a plasticizer, a preservative, a reducing agent, a hydroperoxide removing agent, a detergent, a buffering agent, a pH adjuster, an excipient, a bulking agent, a dispersion agent, a solubilizer, a solidification aid, or a combination thereof.

Antimicrobial Agent

The composition may further comprise a microcidal or antimicrobial agent. As used herein, an “antimicrobial agent” is any compound that slows or stops the growth of a microorganism. In certain embodiments, the inorganic compound kills one or more microbial organism, such as a bacterium, protist, and/or fungus. In certain embodiments, the inorganic compound inhibits the growth of one or more microbial organism, such as a bacterium, protist, virus, or fungus. Suitable antimicrobial agents may include, without limitation, penicillin, cephalosporin, ampicillin, amoxycillin, aztreonam, clavulanic acid, imipenem, streptomycin, gentamycin, vancomycin, clindamycin, polymyxin, erythromycin, bacitracin, amphotericin, nystatin, rifampicin, tetracycline, chlortetracycline, doxycycline, chloramphenicol, ammolfine, butenafine, naftifine, terbinafine, ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, silver sulfadiazine, undecylenate, undecylenic acid, undecylenic alkanolamide, Carbol-Fuchsin, nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, Rutin, Tannic acid, Direct Red 80, Purpurin compounds and analogs, derivatives and salts thereof.

Plasticizer

The composition may additional comprise a plasticizer. As used herein, a “plasticizer” is any agent capable of facilitating or improving the storage function of a dry-state matrix. Thus, in certain embodiments, the plasticizer improves the mechanical properties of a dry-state matrix. In certain embodiments, the plasticizer improves the durability, including resistance to vibrational and other damage, of a dry-state matrix. In certain embodiments, the plasticizer facilitates the reversible dissociation between inorganic compounds and raw sample upon re-hydration of a dry-state matrix. In other embodiments, the plasticizer facilitates the reversible dissociation between stabilizers and raw sample upon re-hydration of a dry-state matrix.

Suitable plasticizers may include polyols such as long-chain polyols, short-chain polyols, and sugars. The plasticizer may include, without limitation, polyvinyl alcohol, polyserine, monosaccharides, disaccharides, complex sugars, ethylene glycol, 1-3 propane diol, glycerol, butane triol (e.g., n-butane triol or isobutane triol), erythritol, pentane triol (e.g., n-pentane triol or isopentane triol), pentane tetraol (e.g., n-pentane tetraol, isopentane tetraol), pentaerythritol, xylitol, sorbitol and mannitol.

Preservatives

The composition may further comprise preservatives used to further prevent the degradation of and damage to the raw sample (and components therein).

Reducing Agents

The composition may additional comprise a reducing agent. Examples of suitable reducing agents include, but are not limited to, cysteine and mercaptoethylene. Examples of metal chelators include, but are not limited to, EDTA, EGTA, o-phenanthroline, dithionite, dithioerythritol, dithiothreitol (DTT), dysteine, 2-mercaptoethanol, mercaptoethylene, bisulfite, sodium metabisulfite, pyrosulfite, pentaerythritol, thioglycolic acid, citrate, urea, uric acid, vitamin C, vitamin E, superoxide dismutases, and analogs, derivatives and salts thereof.

Hydroperoxide Removing Agents

The composition may further comprise a hydroperoxide removing agent. Examples of suitable hydroperoxide removing agents include, but are not limited to, catalase, pyruvate, glutathione, and glutathione peroxidases.

### Raw Sample Material

The raw sample according to the present application generally contains wholly or a high quantity of intact cells, i.e. cells that have not yet been intentionally lysed. Although some cells in a raw sample may be ruptured due to natural causes or the state of the sample upon collection, a raw sample according to the present application does not contain cells intentionally ruptured, or otherwise processed or prepared.

The source of the raw sample may comprise, without limitation, a biological fluid, a biological suspension, a fluid aspirate, blood, plasma, serum, lymph, cerebrospinal fluid, gastric fluid, bile, perspiration, ocular fluid, tears, oral fluid, sputum, saliva, a buccal sample, a tonsil sample, a nasal sample, mucus, a nasopharyngeal sample, semen, urine, a vaginal sample, a cervical sample, a rectal sample, a fecal sample, a wound or purulent sample, hair, a tissue, a tissue homogenate, cells, a cellular lysate, a tissue or cell biopsy, skin cells, tumor or cancer cells, a microbe, a pathogen, a bacterium, a fungus, a protozoan or a virus, or any combination thereof. Preferably the raw sample comprises nucleic acids, including but not limited to, single-stranded and double-stranded polynucleotides containing RNA nucleotides and/or DNA nucleotides. In a preferred embodiment, the raw material comprises RNA; more preferably, the raw sample comprises viral RNA. In a further preferred embodiment, the raw sample comprises one or more nucleic acid types according to the table below.

| Group | Nucleic Acid | Examples | Genome Size (kb) |
|---|---|---|---|
| I | dsDNA | Small Pox | 130-375 |
| | | Herpes | 120-225 |
| | | Adeno | 30-38 |
| | | Papilloma | 8.0 |
| | | Polyoma | 5.3 |
| II | ssDNA | Parvo, Circo | 5.0 |
| III | ss(+)RNA | Corona/SARS | 27-31 |
| | | Hepatitis C. | 10.5 |
| | | Hepatitis A | 7.5 |
| | | Toga | 9.7-11.8 |
| | | Foot & Mouth | 8.5 |
| | | Polio | 7.4 |
| | | TMV | 6.4 |
| IV | ss(−)RNA | Influenza | 12-15 |
| | | Measles | 17-20 |
| VI | ssRNA RT | HIV | 9.75 |
| VII | dsDNA RT | HBV | 3.1 |

In an embodiment, the raw sample is contained within and/or bound by the dry state matrix of the present application. In some embodiments, at least about 50%, 60%, 70%, 80%, 90%, 95%, 99% or 100% of the raw sample by mass is contained within and/or bound by the dry state matrix of the present application. The raw sample contained within and/or bound by the composition of the present application may be stored in a closed container (e.g., a capped tube, vial or well) at a temperature from about −80° C. to about 40° C. for at least about 1 day, 3 days, week, 2 weeks, 3 weeks, 1 month, 2 months, 3 months, 4 months, 1 year, 1.5 years or 2 years.

Surprisingly, raw samples stored and preserved according to the present application are highly resistant to hydrolytic damage, oxidative damage, denaturation (e.g., irreversible unfolding or irreversible loss of secondary structure or tertiary structure), and other mechanical damage. Further, unexpectedly, the raw samples stored and preserved according to the present application have a high retention of function/activity, and demonstrate this retention of activity for up to 2 years.

### Form of the Composition

In an embodiment, the composition is a dry state, such as a dry state matrix. In an embodiment, the components of the composition concentrate upon drying and form a crystalline or paracrystalline structure. In certain embodiments, the composition does not form a glass structure upon drying. As used herein, the term “glass structure” refers to a solid-state structure in which the molecules comprising the glass structure display only short-range order, rather than extended-range crystalline order with respect to one another. In certain embodiments, the components of the composition are capable of co-localization with the raw sample. For example, in certain embodiments, the matrix formed by the components of the composition concentrates upon drying and forms a crystalline or paracrystalline state in direct contact with the cells of the raw sample.

In an embodiment, the composition may be provided as a powder, tablet, pill, or may be carried by a solid support, such as a cotton swab, a filter paper, or a sponge. The composition may also be contained in any suitable container. In a preferred embodiment, the composition and raw sample are carrier by paper, and are stabilized in the form of dried blood spots (DBS) and/or dried plasma spots (DPS).

The composition may be directly added to a raw sample (or vice versa), raw sample/liquid mixture, or present in a collection vessel prior to collection of the raw sample or raw sample/liquid mixture. In some embodiments, the composition added to a raw sample, raw sample/liquid mixture, or other type of raw sample fully solidifies. In some embodiments, composition together with raw sample is fully solidified into a matrix. In other embodiments, the composition added to a raw sample, raw sample/liquid mixture, or other type of raw sample only solidifies partially. The partially solidified composition together with raw sample may form a matrix.

In another embodiment, the composition may be delivered in pre-measured aliquots loaded into sample collection vessels and/or wells, to which an appropriate volume of the raw sample may be added. In such a circumstance, the collection vessels and/or wells are agitated to aid in the even distribution and dispersal of both the composition of the present application and the raw sample.

In a further embodiment, a vial for collecting raw samples can be supplied with pre-measured aliquots of the composition of the present application; an appropriate volume of the raw sample may be subsequently added. Much like the collection vessels and/or wells, the vial is then agitated.

In a still further embodiment, the composition of the present application is provided as part of a kit for collecting samples. The kit may comprise a composition according to the present application, a raw sample, a carrier comprising a container or solid support for the composition and raw sample, and instructions for using the kit for the stabilization and storage of a given raw sample. The kits according to the present application may be adapted for shipment by mail. For example, in addition to the composition, raw sample, carrier, and instructions, the kit may comprise closures for closing/sealing the carrier from contamination (such as tape, a sealable bag, a cap, a stopper, or other sealant material), an additional container (comprising a box, flexible pouch, envelope, etc.) for receiving and transporting the carrier, a pre-addressed mailing label, and a protective or cushioning material such as protective foam, packing peanuts, and/or shredded paper filler, etc. Significantly, the system of the present application effectively stabilizes raw samples such that the samples do not need to be refrigerated or frozen during shipping or storage.

### Methods of Preparation, Storing, and Preserving

In an embodiment, the compositions of the present application can be prepared by mixing one or more metal chelators, a hydroxyl radical scavenger, a singlet oxygen quencher, and an RNase inhibitor together with a cell separation reagent, and transferring the resulting mixture to a carrier.

In an embodiment, a raw sample may be stabilized and stored at room temperature for up to 2 years by providing the composition of the present application, collecting one or more raw samples, mixing the one or more raw samples with the composition of the present application, and optionally allowing the mixture to dry. In some embodiments, the mixture will form a matrix. The mixture may be wholly solid, or solid in part.

In a further embodiment, after stabilization and storage for a desired period of time, the raw sample bound in/by the composition of the present application may be rehydrated by the addition of an aqueous solution (e.g., water or an aqueous buffer) shortly before the composition is to be used in a biochemical reaction (e.g., PCR) or an analysis (e.g., an immunoassay).

In an embodiment, the compositions of the present application as provided in a kit may be used by providing the composition of the present application in a carrier, collecting one or more raw samples, mixing the one or more raw samples with the composition in a carrier, sealing the mixture in the carrier with closures, placing the sealed mixture in an additional container, adding protective materials to the additional container, and applying a pre-addressed mailing label to the additional container.

In a still further embodiment, the composition of the present application may be used as part of automated and/or high throughput preparation, stabilization, and storage of raw samples.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

The compositions of the present application were evaluated for their ability to stabilize HIV-1 virus present in both whole blood and plasma on paper. The whole blood and plasma were provided in a solid state, in the form of dried plasma spots (DPS) and dried blood spots (DBS). To further evaluate the effect of PEG as a cell separation reagent, various weight percentages of PEG-600 were evaluated. In particular, PEG-600 was evaluated at weight percentages of 15%, 50%, and 80% in the stabilization of DPS. For DBS, PEG was evaluated at 12.5%, 15%, 31.25%, 50% and 80% PEG.

The effect of PEG in the formulations of the present application were compared to a control comprising the other components of composition according to the application but excluding PEG, and to a comparative formula using Guanidium chloride (GuHCl), a denaturing chaotropic agent, in place of PEG. These formulations can be shown in Table 1 below, wherein Formulation 1 represents the formulation according to the present application, Comparative Composition A represents the control lacking PEG, and Comparative Composition B represents the formula using GuHCl.

TABLE 1

| | | | Comparative | | Comparative | |
|---|---|---|---|---|---|---|
| Material | Formulation 1 | | Composition A | | Composition B | |
| Citric Acid | 188.06 | mM | 188.06 | mM | 188.06 | mM |
| EDTA | 1.5 | mM | 1.5 | mM | 1.5 | mM |
| Mannitol | 206 | mM | 206 | mM | 206 | mM |
| Cysteine | 188.06 | mM | 188.06 | mM | 188.06 | mM |
| ATA | 3.0 | mM | 3.0 | mM | 3.0 | mM |
| PEG-600 mM | 50 | wt. % | | | | |
| Guanidium | | | | | 1.6 | mM |
| Chloride | | | | | | |

The evaluation was conducted by spiking 210000 copies/mL (5.3_{log10}) of HIV-1 in either 30 uL of whole blood or 30 uL of plasma. The samples were applied to paper, and the solid-state samples were dried for 3 hours at room temperature and then stressed for 3 hours at 72° C. The stressed conditions are equivalent to approximately 4 days at ambient temperature. All samples were then analyzed on the COBAS® TaqMan® HIV-1 Test. The stabilization and storage effectiveness are expressed in terms of percent recovery.

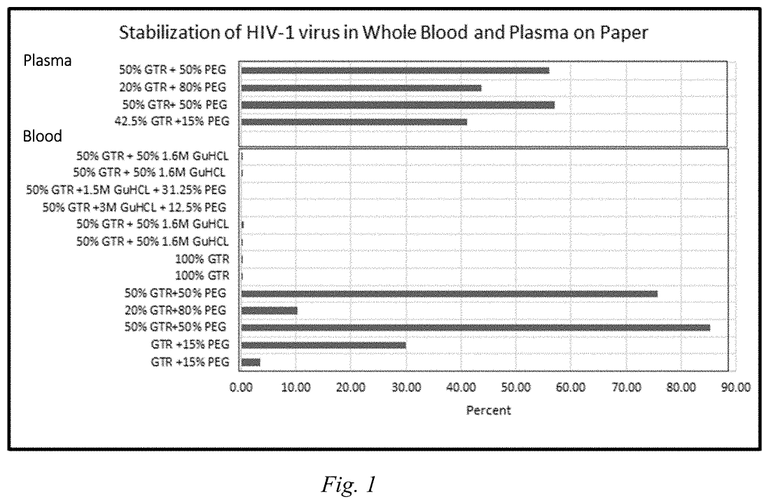

The results of this analysis are shown in FIG. 1. FIG. 1 demonstrates good recovery of viral RNA in DPS for concentrations of PEG ranging from 15% to 80%. The improvement in recovery is even more pronounced for DBS, where the recovery of viral RNA approaches 90%. The recovery of HIV from both DPS and DBS is significantly improved over both Comparative Compositions A and B. Both comparative compositions demonstrated virtually 0% recovery. These results indicate the significant and surprising role of PEG in stabilizing and storing nucleic acids, and particularly viral RNA.

### Example 2

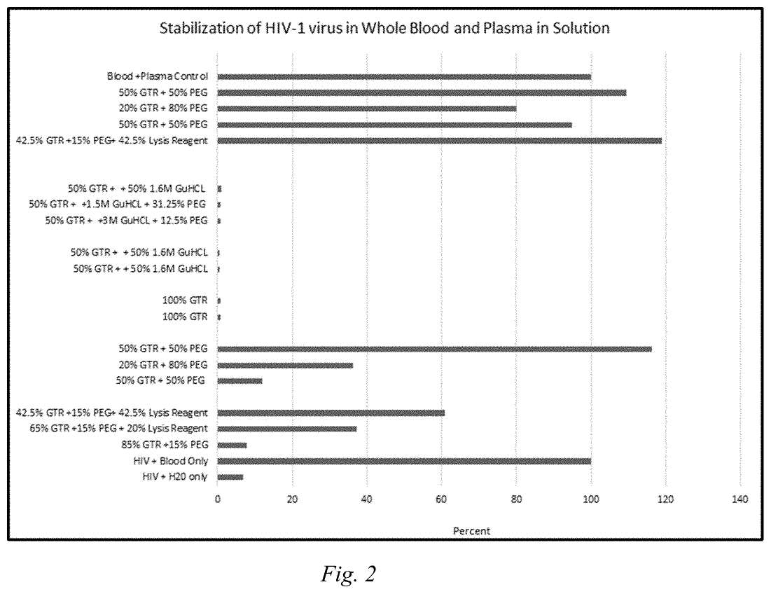

The experimental procedures of Example 1 were repeated, except that the stabilization of HIV-1 virus was evaluated for whole blood and plasma in solution. In addition to the formulations and compositions in Table 1, further controls were added. The additional controls in this case were a solution of HIV-1 virus and blood only, the HIV-1 virus and water only, and finally the HIV-1 virus and plasma only. These controls contained 210,000 copies/mL of the HIV virus, spiked in 30 uL whole blood, plasma, or water. These controls were stored at −80° C., mimicking currently existing storage procedures. Like Example 1, the preservation and storage efficacy are expressed in terms of percent recovery. The results of this evaluation are shown in FIG. 2.

FIG. 2 shows that for samples stored in solution, the presence of PEG is important for the successful storage and recovery of the HIV-1 virus in solution.

### Example 3

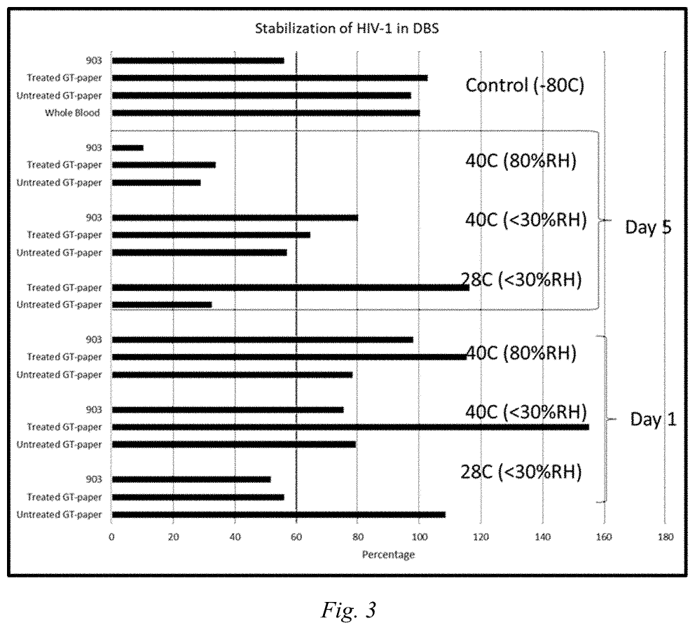

The test procedures of Example 1 were repeated, except that the samples evaluated were 903, paper treated according to formulation 1 of table 1, untreated paper, and whole blood. These samples where then applied to paper and dried as DBS samples according to Example 1. The DBS samples were dried at 72 C for 2 hours followed by storage for 1 day and 5 days at either 40 C with 80% relative humidity, 40 C with <30% relative humidity or at ambient temperature of 28 C with <30% relative humidity. Representative control DBS samples on 903 paper, untreated GT-paper and GT-paper treated with formulation 1 and control liquid whole blood samples were stored at −80 C. At the end of the 1 day and 5 day incubation periods, the experimental and control DBS samples were rehydrated with nuclease free water to the original volume of the sample applied to the DBS and analyzed with the COBAS® TaqMan® HIV-1 Test. The results of this evaluation are shown in FIG. 3.

FIG. 3 shows that the treated paper according to the present application results in substantially improved recovery of the HIV-1 virus. In particular, the storage methods of the present application performed just as well as and often better than the controls. Recovery of HIV-1 material on treated paper ranged from just under 60% up to approximately 155%. The increased recovery is attributed to the increased drying time of the DBS and the enhancement of the PCR reaction by the chemicals in particular the PEG in formulation 1. Further, the storage methods of the present application demonstrate the ability to stabilize and store raw samples in GT-paper treated with formulation 1 for up to 5 days under extreme conditions of high temperature and high relative humidity of 40° C. and >80% humidity whereas the untreated GT-paper or 903 paper failed.

The inventions being thus described, it will be obvious that the same may be varied in many ways. Such variations are not to be regarded as a departure from the spirit and scope of the inventions and all such modifications are intended to be included within the scope of the following claims.

The above specification provides a description of the manufacture and use of the disclosed compositions and methods. Since many embodiments can be made without departing from the spirit and scope of the invention, the invention resides in the claims.

What is claimed is:

1. A composition for stabilizing and storing viral RNA comprising:
one or more metal chelators;
a hydroxyl radical scavenger;
a singlet oxygen quencher;
an RNase inhibitor; and
a cell separation reagent.

2. The composition of claim 1, wherein the composition comprises two metal chelators.

3. The composition of claim 2, wherein the two metal chelators comprise citric acid and an aminocarboxylate.

4. The composition of claim 1, wherein the hydroxyl radical scavenger comprises mannitol.

5. The composition of claim 1, wherein the singlet oxygen quencher comprises cysteine.

6. The composition of claim 1, wherein the RNase inhibitor comprises ATA.

7. The composition of claim 1, wherein the cell separation reagent comprises a polyethylene glycol.

8. The composition of claim 1, wherein the composition stabilizes and stores viral RNA for at least five days.

9. The composition of claim 1, wherein the composition stabilizes and stores viral RNA at a temperature between about 20° C. and about 60° C.

10. The composition of claim 1, wherein the viral RNA is provided as part of a sample of whole blood or blood plasma.

11. The composition of claim 10, wherein the viral RNA is stored on a paper carrier as dried blood spots (DBS) or dried plasma spots (DPS).

12. A kit for stabilizing and storing viral RNA comprising:
a composition comprising one or more metal chelators, a hydroxyl radical scavenger, a singled oxygen quencher, an RNase inhibitor, and a cell separation reagent;
one or more carriers; and
one or more closures;
wherein the composition is combined with a sample comprising viral RNA, held in the one or more carriers, and sealed by the one or more closures; and
wherein the composition stabilizes viral RNA for at least five days, and wherein the composition stabilizes viral RNA at an ambient temperature.

13. The kit of claim 12, wherein the one or more carriers comprises one or more vials, one or more wells, paper, and/or a cotton swab.

14. The kit of claim 13, further comprising an additional container for housing the composition and sample held in the one or more carriers and sealed by the one or more closures.

15. The kit of claim 14 wherein the additional container comprises a box and/or an envelope.

16. The kit of claim 15, further comprising a pre-addressed mailing label.

17. A method of using a kit for stabilizing and storing viral RNA comprising:
providing a composition comprising one or more metal chelators, a hydroxyl radical scavenger, a singled oxygen quencher, an RNase inhibitor, and a cell separation reagent, wherein the composition stabilizes viral RNA for at least five days, and wherein the composition stabilizes viral RNA at an ambient temperature;
collecting one or more raw samples;
mixing the one or more raw samples with the composition in one or more carriers; and
sealing the mixture in the carrier with closures.

18. The method of claim 17, further comprising the step of placing the sealed mixture in an additional container for housing the one or more carriers.

19. The method of claim 18, further comprising the step of adding protective materials to the additional container, wherein the protective materials comprise protective foam, packing peanuts, and/or shredded paper filler

20. The method of claim 19, further comprising the step of applying a pre-addressed mailing label to the additional container.
